(19) United States
(12) Patent Application Publication
(10) Pub. No.: US 2024/0208887 A1
(43) Pub. Date: Jun. 27, 2024

(54) METHOD FOR PREPARING 2,3,3,3-TETRAFLUOROPROPENE

(71) Applicants: ZHEJIANG RESEARCH INSTITUTE OF CHEMICAL INDUSTRY CO., LTD, Hangzhou, Zhejiang (CN); SINOCHEM LANTIAN CO., LTD., Hangzhou, Zhejiang (CN)

(72) Inventors: Minyang LIU, Hangzhou, Zhejiang (CN); Wanjin YU, Hangzhou, Zhejiang (CN); Shengda LIN, Hangzhou, Zhejiang (CN); Xinbao XIAO, Hangzhou, Zhejiang (CN); Xia LUO, Hangzhou, Zhejiang (CN); Wucan LIU, Hangzhou, Zhejiang (CN); Jianjun ZHANG, Hangzhou, Zhejiang (CN)

(21) Appl. No.: 18/286,137

(22) PCT Filed: Apr. 7, 2022
(86) PCT No.: PCT/CN2022/085539

§ 371 Date: Oct. 9, 2023

(30) Foreign Application Priority Data
Apr. 15, 2021 (CN) 202110404614.6
Apr. 15, 2021 (CN) 202110404622.0
Apr. 15, 2021 (CN) 202110404625.4

(51) Int. Cl. C07C 17/278; B01J 21/18; B01J 23/26; B01J 23/42; B01J 23/44; B01J 27/125; B01J 27/135; B01J 27/138; B01J 27/32; B01J 31/26; B01J 31/38; B01J 37/00; B01J 37/02; B01J 37/04; B01J 37/18; C07C 17/25

(52) U.S. Cl. CPC C07C 17/278; B01J 21/18; B01J 23/26; B01J 23/42; B01J 23/44; B01J 27/125; B01J 27/135; B01J 27/138; B01J 27/32; B01J 31/26; B01J 31/38; B01J 37/0009; B01J 37/0207; B01J 37/0236; B01J 37/04; B01J 37/18; C07C 17/25; B01J 2231/32

(57) ABSTRACT

Disclosed in the present disclosure is a method for preparing 2,3,3,3-tetrafluoropropene. The method includes a two-step method for preparing 2,3,3,3-tetrafluoropropene, a method for co-producing 2,3,3,3-tetrafluoropropene and 1-chloro-2,3,3,3-tetrafluoropropene, and a method for co-producing 2,3,3,3-tetrafluoropropene and 1-chloro-3,3,3-trifluoropropene. The two-step method for preparing 2,3,3,3-tetrafluoropropene includes: A1, a telomerization step: subjecting chlorofluoromethane and trifluoroethylene to a pressure telomerization reaction under the action of a telomerization catalyst to prepare 3-chloro-1,1,1,2-tetrafluoropropane, wherein the telomerization catalyst is a Lewis acid catalyst or a mixed catalyst of a Lewis acid catalyst and dichloromethane; and A2, a dehydrochlorination step: subjecting the 3-chloro-1,1,1,2-tetrafluoropropane to dehydrochlorination under the catalytic action of activated carbon to obtain 2,3,3,3-tetrafluoropropene. The method for preparing 2,3,3,3-tetrafluoropropene has the advantages of a simple process, high product selectivity, mild reaction conditions and the like.

### TECHNICAL FIELD

The present disclosure relates to preparation of 2,3,3,3-tetrafluoropropene. In particular to a method for preparing 2,3,3,3-tetrafluoropropene by reactions of telomerization and removal (dehydrochlorination, dehydrofluorination, dehydrogenation and the like) In two steps with trifluoroethylene as a raw material.

### BACKGROUND

2,3,3,3-tetrafluoropropene has an ozone depression potential (ODP) value of zero, a global warming potential (GWP) value of less than 1, lower life cycle climate performance (LCCP) than a traditional refrigerant HFC-134a, better system refrigeration performance than the HFC-134a and same atmospheric decomposition products as the HFC-134a. Thus, the 2,3,3,3-tetrafluoropropene is considered as the most promising substitute for automotive refrigerants at present, and has been accepted by many major automobile manufacturers. At present, the 2,3,3,3-tetrafluoropropene has the following preparation routes.

### 1. Hexafluoropropene Route:

The 2,3,3,3-tetrafluoropropene is prepared by reactions in four steps with hexafluoropropene as a raw material: (1) subjecting hexafluoropropene and hydrogen to a hydrogenation reaction to prepare 1,1,1,2,3,3-hexafluoropropane (HFC-236ea); (2) subjecting the HFC-236ea to a dehydrofluorination reaction under the action of a catalyst to prepare 1,1,1,2,3-pentafluoropropene (HFO-1225ye); (3) subjecting the HFO-1225ye and hydrogen to a hydrogenation reaction to prepare 1,1,1,2,3-pentafluoropropane (HFC-245eb); and (4) subjecting the HFC-245eb to a dehydrofluorination reaction under the action of a catalyst to prepare the 2,3,3,3-tetrafluoropropene.

In a United States patent US20070179324A and Chinese patents CN101544536A, CN10226789A, CN102026947A and the like, methods for preparing 2,3,3,3-tetrafluoropropene by reactions of hydrogenation, dehydrofluorination, re-hydrogenation and re-dehydrofluorination in four steps with hexafluoropropene as a raw material are disclosed. These methods have the characteristics of a simple process, a mature technology and the like, but have the problems of many reaction steps, a variety of intermediate products requiring separation and purification, complicated process steps, large investment in equipment, low reaction yield, high separation cost, high energy consumption and the like.

In order to overcome the shortcomings of technologies in the above patents, a Chinese patent CN103449963B discloses a method for synthesizing 2,3,3,3-tetrafluoropropene by continuous reactions in multiple steps with hexafluoropropene as a raw material. Continuous production based on direct reactions of intermediate products, such as the HFC-236ea, the HFO-1225ye and the HFC-245eb, without separation can be realized. However, without separation and purification of the intermediate products, it is indicated that impurities will be accumulated and increased in reaction materials constantly, the yield of the target product 2,3,3,3-tetrafluoropropene is eventually affected, and meanwhile, difficulty in rectification and separation of a 2,3,3,3-tetrafluoropropene product is increased.

### 2. Tetrachloropropene (TCP) Route:

A patent CN101395108B discloses a method for preparing 2,3,3,3-tetrachloropropene by reactions in three steps with 1,1,2,3-tetrachloropropene as a raw material. The method includes the following reaction steps: (1) subjecting 1,1,2,3-tetrachloropropene and hydrogen fluoride (HF) to a gas phase fluorination reaction to prepare 2-chloro-3,3,3-trifluoropropene (HCFO-1233xf) with a selectivity of 80-96%, wherein when Cr₂O₂ and FeCl₂/AC catalysts are used, the selectivity reaches 96%, and the conversion rate is only 20%; (2) subjecting the HCFO-1233xF and HF to an addition reaction with SbCl₅ as a catalyst to produce 2-chloro-1,1,1,2-tetrafluoropropane (HCFC-244bb); and (3) subjecting the HCFC-244bb to a gas phase dehydrochlorination reaction under the action of an activated carbon catalyst to prepare the target product 2,3,3,3-tetrafluoropropene. The process is complex in reaction steps and unfavorable to industrial production, and has the problems of low conversion rate, high reaction temperature and the like.

A United States patent US20090099396 discloses a method for preparing 2,3,3,3-tetrafluoropropene by reactions in two steps with 1,1,2,3-tetrachloropropene as a raw material. The method includes the following reaction steps' (1) subjecting 1,1,2,3-tetrachloropropene and HF to a liquid phase fluorination reaction with SbCl₅ as a catalyst to prepare 1,1,1,2,3-pentafluoropropane (HFC-245eb), wherein the conversion rate of TCP can reach 100%, but the selectivity of HFC-245eb is only 53-50%, and many by-products are produced; and (2) subjecting the HFC-245eb to a liquid phase dehydrofluorination reaction under the action of an alkali metal hydroxide to produce the target product 2,3,3,3-tetrafluoropropene. The process has the advantages of few reaction steps and low investment in equipment. However, the intermediate product HFC-245eb has low selectivity, and the by-products are difficult to separate

### 3. Trifluoropropene Route:

A patent CN101979364A discloses a method for preparing 2,3,3,3-tetrafluoropropene with 3,3,3-trifluoropropene as a raw material. The method includes the following four reaction steps: (1) subjecting 3,3,3-trifluoropropene and chlorine to an addition reaction under the action of photocatalysis to produce 1,2-dichloro-3,3,3-trifluoropropane, wherein the conversion rate of the raw material reaches 95%, and the selectivity reaches 90%; (2) subjecting the 1,2-dichloro-3,3,3-trifluoropropane to a liquid phase dehydrochlorination reaction under the action of an alkali metal hydroxide to produce 2-chloro-3,3,3-trifluoropropene (HCFO-1233xf), wherein the conversion rate and the selectivity both reach 90%; (3) subjecting the HCFO-1233xf and HF to an addition reaction to produce 2-chloro-1,1,1,2-tetrafluoropropane (HCFC-244bb), wherein a catalyst is SnCl₄, TiCl₄ or fluorosulfonic acid, the conversion rate of the raw material reaches 95%, and the selectivity is 90-98%; and (4) subjecting the HCFC-244bb to a liquid phase dehydrochlorination reaction under the action of an alkali metal catalyst to prepare a target product CF₃CF═CH₂, wherein the conversion rate of the raw material is 95%, and the selectivity is 90-95%. The process has a long synthetic route, a chlorination reaction in the first step has high requirements for equipment, more waste liquid is produced by dehalogenation reactions in two steps, and the reactions have a low total yield and a high synthetic cost.

### 4. Other Routes:

An Asahi patent WO2011162341A discloses a method for preparing 2,3,3,3-tetrafluoropropene by hydrogenation reduction with 1,1-dichloro-2,3,3,3-tetrafluoropropene (CFO-1214ya) as a raw material under the action of a palladium catalyst. However, by means of the method, the hydrogenation reduction reaction degree is difficult to control, and intermediates or over-reduction products such as 1-chloro-2,3,3,3-tetrafluoropropene (HCFO-1224yd), 1-chloro-2,3,3,3-tetrafluoropropane (HCFC-244eb) and 2,3,3,3-tetrafluoropropane (HFC-254eb) are easily produced. The method has low product selectivity and complex post-treatment operations. In addition, the by-product HFC-254eb is prone to a further dehydrofluorination reaction during alkali washing treatment to produce 3,3,3-trifluoropropene (HFO-1243zf) having a boiling point similar to that of HFO-1234yf, thereby further increasing the difficulty in separation of impurities. Although the above problems can be reduced by controlling the reaction temperature of a catalyst bed layer and the absorption temperature of alkali washing, the problems are not reduced obviously. Moreover, process conditions are difficult to control, and the method is not suitable for industrial amplification.

### SUMMARY

In order to solve the above technical problems, the present disclosure provides a two-step method for preparing 2,3,3,3-tetrafluoropropene, which has a simple process, mild reaction conditions and high product selectivity and is suitable for industrial production.

The object of the present disclosure is realized through the following technical schemes.

The present disclosure provides a two-step method for preparing 2,3,3,3-tetrafluoropropene. The method includes:
A1, a telomerization step: subjecting chlorofluoromethane and trifluoroethylene to a pressure telomerization reaction under the action of a telomerization catalyst to prepare 3-chloro-1,1,1,2-tetrafluoropropane, wherein the telomerization catalyst is a Lewis acid catalyst or a mixed catalyst of a Lewis acid catalyst and dichloromethane; and
A2, a dehydrochlorination step: subjecting the 3-chloro-1,1,1,2-tetrafluoropropane to dehydrochlorination under the catalytic action of activated carbon to obtain 2,3,3-tetrafluoropropene.

The two-step method for preparing 2,3,3,3-tetrafluoropropene in the present disclosure has a reaction equation as follows:

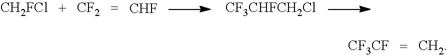

The Lewis acid catalyst of the present disclosure is selected from at least one halide of Al, Sb, Ti, Zr and Hf. As a preference, the Lewis acid catalyst is selected from at least one of ZrCl₄, HfCl₄, TiCl₄, AlCl₃, AlF₃ and SbF₅. More preferably, the Lewis acid catalyst is ZrCl₄ or HfCl₄.

The raw materials, chlorofluoromethane and trifluoroethylene, of the present disclosure are subjected to the telomerization reaction under pressure conditions, and the raw material, chlorofluoromethane, is partially or completely converted into liquid under reaction conditions. In addition, the 3-chloro-1,1,1,2-tetrafluoropropane produced by the telomerization reaction is liquid. Therefore, a solvent-free reaction is preferably used in the step A1 of the present disclosure to reduce a separation step of an intermediate and/or a product.

The telomerization catalyst of the present disclosure may be a single Lewis acid catalyst or a mixed catalyst of a Lewis acid catalyst and dichloromethane. When a mixed catalyst is used, the Lewis acid catalyst dissociates and activates the chlorofluoromethane to form F⁺, CH₂Cl⁺, Cl⁻, CH₂F⁺ and other ions; and the dichloromethane inhibits the dissociated F⁺, CH₂Cl⁺, Cl⁻, CH₂F⁺ and other ions from rebinding, so as to ensure that the F⁻ and CH₂Cl⁺ ions undergo a directed telomerization reaction with the trifluoroethylene to obtain a telomerization product CF₃CHFCH₂Cl with high selectivity.

In a chemical reaction, the ratio of raw materials, the ratio of raw materials to a catalyst, the reaction temperature, the reaction time and the like will affect reaction results. In particular, combinations of multiple variables will have a great impact on reaction results.

In the telomerization step of the present disclosure, the molar ratio of the chlorofluoromethane to the trifluoroethylene is 1.0.1 to 1:10; and more preferably, the molar ratio of the chlorofluoromethane to the trifluoroethylene is 1:1 to 1:5. The amount of the Lewis acid catalyst is 0.01 to 50 wt % of the mass of the chlorofluoromethane; and more preferably, the amount of the Lewis acid catalyst is 0.1 to 10 wt % of the mass of the chlorofluoromethane. When a mixed catalyst of a Lewis acid catalyst and dichloromethane is used, the molar ratio of the dichloromethane to the chlorofluoromethane is 1:0.01 to 1:10; and more preferably, the molar ratio of the dichloromethane to the chlorofluoromethane is 1:0.1 to 1:5.

The telomerization step of the present disclosure is carried out under pressure conditions at a reaction temperature of −30° C. to 100° C. and a reaction pressure of 0.5 to 5.0 MPa for a reaction time of 1 to 50 h. More preferably, the reaction temperature is 0 to 50° C., the reaction pressure is 0.8 to 3.0 MPa, and the reaction time is 5 to 10 h.

The dehydrochlorination step of the present disclosure is carried out under the catalytic action of activated carbon, and the activated carbon is selected from fruit shell type activated carbon, coal type activated carbon or wood type activated carbon and is preferably fruit shell type activated carbon.

The dehydrochlorination step is carried out at a reaction temperature of 200 to 500° C., and preferably, the reaction temperature is 300-350° C.

In order to further improve the purity of a 2,3,3,3-tetrafluoropropene product and reduce post-treatment difficulty, the 3-chloro-1,1,1,2-tetrafluoropropane obtained in the telomerization step is used in the dehydrochlorination step after rectification and separation.

In a second aspect, the present disclosure provides a method for co-producing 2,3,3,3-tetrafluoropropene and 1-chloro-2,3,3,3-tetrafluoropropene. The method for co-producing 2,3,3,3-tetrafluoropropene and 1-chloro-2,3,3,3-tetrafluoropropene includes:
A1, a telomerization step: subjecting chlorofluoromethane and trifluoroethylene to a pressure telomerization reaction under the action of a telomerization catalyst to prepare 3-chloro-1,1,1,2-tetrafluoropropane, wherein the telomerization catalyst is a Lewis acid catalyst or a mixed catalyst of a Lewis acid catalyst and dichloromethane; and
A2, a removal step: subjecting the 3-chloro-1,1,1,2-tetrafluoropropane to a dehydrochlorination reaction and a dehydrogenation reaction simultaneously under the action of an activated carbon supported noble metal catalyst to obtain 2,3,3,3-tetrafluoropropene and 1-chloro-2,3,3,3-tetrafluoropropene, wherein the activated carbon supported noble metal catalyst is at least one of Pd/activated carbon (Pd/AC) and Pt/activated carbon (Pt/AC).

The method for co-producing 2,3,3,3-tetrafluoropropene and 1-chloro-2,3,3,3-tetrafluoropropene in the present disclosure has a reaction equation as follows:

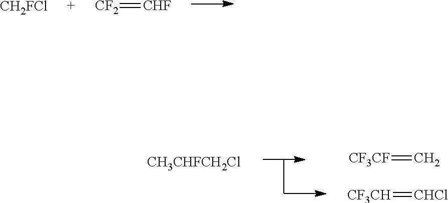

The Lewis acid catalyst of the present disclosure is selected from at least one halide of Al, Sb, Ti, Zr and Hf. As a preference, the Lewis acid catalyst is selected from at least one of ZrCl₄, HfCl₄, TiCl₄, AlF₃, AlCl₃ and SbF₅. More preferably, the Lewis acid catalyst is ZrCl₄ or HfCl₄.

The raw materials, chlorofluoromethane and trifluoroethylene, of the present disclosure are subjected to the telomerization reaction under pressure conditions, and the raw material, chlorofluoromethane, is partially or completely converted into liquid under reaction conditions. In addition, the 3-chloro-1,1,1,2-tetrafluoropropane produced by the telomerization reaction is liquid. Therefore, a solvent-free reaction is preferably used in the step A1 of the present disclosure to reduce a separation step of an intermediate and/or a product.

The telomerization catalyst of the present disclosure may be a single Lewis acid catalyst or a mixed catalyst of a Lewis acid catalyst and dichloromethane. When a mixed catalyst is used, the Lewis acid catalyst dissociates and activates the chlorofluoromethane to form F⁻, CH₂Cl⁺, Cl⁻, CH₂F⁺ and other ions; and the dichloromethane inhibits the dissociated F⁻, CH₂Cl⁺, Cl⁻, CH₂F⁺ and other ions from rebinding, so as to ensure that the F⁻ and CH₂Cl⁺ ions undergo a directed telomerization reaction with the trifluoroethylene to obtain a telomerization product CF₃CHFCH₂Cl with high selectivity.

In a chemical reaction, the ratio of raw materials, the ratio of raw materials to a catalyst, the reaction temperature, the reaction time and the like will affect reaction results. In particular, combinations of multiple variables will have a great impact on reaction results.

In the telomerization step of the present disclosure, the molar ratio of the chlorofluoromethane to the trifluoroethylene is 1:0.1 to 1:10; and more preferably, the molar ratio of the chlorofluoromethane to the trifluoroethylene is 1:1 to 1:5. The amount of the Lewis acid catalyst is 0.01 to 50 wt % of the mass of the chlorofluoromethane; and more preferably, the amount of the Lewis acid catalyst is 0.1 to 10 wt % of the mass of the chlorofluoromethane. When a mixed catalyst of a Lewis acid catalyst and dichloromethane is used, the molar ratio of the dichloromethane to the chlorofluoromethane is 1:0.01 to 1:10; and more preferably, the molar ratio of the dichloromethane to the chlorofluoromethane is 1:0.1 to 1:5.

The telomerization step of the present disclosure is carried out under pressure conditions at a reaction temperature of −30° C. to 100° C. and a reaction pressure of 0.5 to 5.0 MPa for a reaction time of 1 to 50 h. More preferably, the reaction temperature is 0 to 50° C., the reaction pressure is 0.8 to 3.0 MPa, and the reaction time is 5 to 10 h.

In the removal step of the present disclosure, under the action of the activated carbon supported noble metal catalyst, the raw material, 3-chloro-1,1,1,2-tetrafluoropropane, is subjected to the dehydrochlorination reaction when adsorbed to activated carbon and is subjected to the dehydrogenation reaction when adsorbed to a noble metal site on the activated carbon, so as to obtain the 2,3,3,3-tetrafluoropropene and the 1-chloro-2,3,3,3-tetrafluoropropene at the same time.

The activated carbon supported noble metal catalyst may be prepared by a conventional method in a case that the activated carbon supported noble metal catalyst of the present disclosure can be obtained. As a preference, the activated carbon supported noble metal catalyst of the present disclosure is prepared by an impregnation method. The impregnation method includes the following steps:
B1, pretreatment of a carrier: drying activated carbon at 90 to 120° C. for 12 h or above,
B2, impregnation in a metal salt: impregnating the pretreated activated carbon in a soluble salt solution of Pd or Pt under vacuum or atmospheric pressure conditions;
B3, drying the impregnated activated carbon at a temperature of 90 to 120° C. for 12 h or above; and
B4, reducing the dried activated carbon by a mixed gas of hydrogen and nitrogen to obtain the activated carbon supported noble metal catalyst, wherein the hydrogen has a volume ratio of 5 to 50% in the mixed gas of hydrogen and nitrogen, and the reducing is carried out at a temperature of 150 to 300° C.

In the activated carbon supported noble metal catalyst, the Pd or the Pt has a supporting capacity of 0.1 to 5.0 wt %, preferably 0.5-1.5 wt %.

A gas-solid reaction is carried out in the removal step of the present disclosure, the 3-chloro-1,1,1,2-tetrafluoropropane is vaporized and then loaded onto a catalyst bed layer by nitrogen to carry out a removal reaction, the removal reaction has a material volume space velocity of 50 to 300 h⁻¹, and the volume ratio of N₂ to the 3-chloro-1,1,1,2-tetrafluoropropane is (0.5-3.0):1, preferably (1.5-2.0):1.

The removal step of the present disclosure is carried out at a reaction temperature of 300 to 600° C. and preferably, the reaction temperature is 400-450° C.

Distribution of products obtained in the removal step A2 may be adjusted in a certain range by adjusting the preparation process of the activated carbon supported noble metal catalyst, the supporting capacity of a noble metal in the catalyst and reaction conditions. Generally, 30-90% of the 2,3,3,3-tetrafluoropropene and 10 to 50% of the 1-chloro-2,3,3,3-tetrafluoropropene are obtained in the removal step A2; and preferably, products obtained in the removal step include 50-60% of the 2,3,3,3-tetrafluoropropene, 30 to 50% of the 1-chloro-2,3,3,3-tetrafluoropropene and remaining by-products, such as 1-chloro-3,3,3-trifluoropropene.

In order to further reduce post-treatment difficulty, the 3-chloro-1,1,1,2-tetrafluoropropane obtained in the telomerization step is used in the removal step after rectification and separation.

In a third aspect, the present disclosure further provides a method for co-producing 2,3,3,3-tetrafluoropropene and 1-chloro-3,3,3-trifluoropropene. The method for co-producing 2,3,3,3-tetrafluoropropene and 1-chloro-3,3,3-trifluoropropene includes:
A1, a telomerization step: subjecting chlorofluoromethane and trifluoroethylene to a pressure telomerization reaction under the action of a telomerization catalyst to prepare 3-chloro-1,1,1,2-tetrafluoropropane, wherein the telomerization catalyst is a Lewis acid catalyst or a mixed catalyst of a Lewis acid catalyst and dichloromethane; and
A2, a dehydrohalogenation step: subjecting the 3-chloro-1,1,1,2-tetrafluoropropane to a dehydrochlorination reaction and a dehydrofluorination reaction simultaneously under the action of a composite dehalogenation catalyst to obtain 2,3,3,3-tetrafluoropropene and 1-chloro-3,3,3-trifluoropropene, wherein the composite dehalogenation catalyst is prepared from at least one oxide or fluoride of Al, Mg or Cr and activated carbon powder.

The at least one oxide or fluoride of Al, Mg or Cr is selected from at least one of Al₂O₃, AlF₃, MgF₂ and Cr₂O₃; and the activated carbon powder is selected from fruit shell type activated carbon, coal type activated carbon or wood type activated carbon.

The method for co-producing 2,3,3,3-tetrafluoropropene and 1-chloro-3,3,3-trifluoropropene in the present disclosure has a reaction equation as follows:

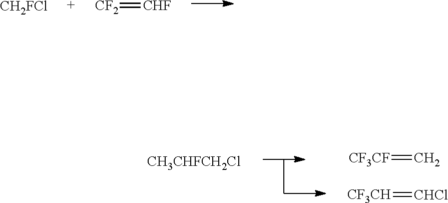

The Lewis acid catalyst of the present disclosure is selected from at least one halide of Al, Sb, Ti, Zr and Hf. As a preference, the Lewis acid catalyst is selected from at least one of ZrCl₄, HfCl₄, TiCl₄, AlF₃, AlCl₃ and SbF₅. More preferably, the Lewis acid catalyst is ZrCl₄ or HfCl₄.

The raw materials, chlorofluoromethane and trifluoroethylene, of the present disclosure are subjected to the telomerization reaction under pressure conditions, and the raw material, chlorofluoromethane, is partially or completely converted into liquid under reaction conditions. In addition, the 3-chloro-1,1,1,2-tetrafluoropropane produced by the telomerization reaction is liquid. Therefore, a solvent-free reaction is preferably used in the step A1 of the present disclosure to reduce a separation step of an intermediate and/or a product.

The telomerization catalyst of the present disclosure may be a single Lewis acid catalyst or a mixed catalyst of a Lewis acid catalyst and dichloromethane. When a mixed catalyst is used, the Lewis acid catalyst dissociates and activates the chlorofluoromethane to form F⁻, CH₂Cl⁺, Cl⁻, CH₂F⁺ and other ions; and the dichloromethane inhibits the dissociated F⁻, CH₂Cl⁺, Cl⁻, CH₂F⁺ and other ions from rebinding, so as to ensure that the F⁺ and CH₂Cl⁺ ions undergo a directed telomerization reaction with the trifluoroethylene to obtain a telomerization product CF₃CHFCH₂Cl with high selectivity.

In a chemical reaction, the ratio of raw materials, the ratio of raw materials to a catalyst, the reaction temperature, the reaction time and the like will affect reaction results. In particular, combinations of multiple variables will have a great impact on reaction results.

In the telomerization step of the present disclosure, the molar ratio of the chlorofluoromethane to the trifluoroethylene is 1:0.1 to 1:10; and more preferably, the molar ratio of the chlorofluoromethane to the trifluoroethylene is 1:1 to 1.5. The amount of the Lewis acid catalyst is 0.01 to 50 wt % of the mass of the chlorofluoromethane; and more preferably, the amount of the Lewis acid catalyst is 0.1 to 10 wt % of the mass of the chlorofluoromethane. When a mixed catalyst of a Lewis acid catalyst and dichloromethane is used, the molar ratio of the dichloromethane to the chlorofluoromethane is 1:0.01 to 1:10; and more preferably, the molar ratio of the dichloromethane to the chlorofluoromethane is 1:0.1 to 1:5.

The telomerization step of the present disclosure is carried out under pressure conditions at a reaction temperature of −30° C. to 100° C. and a reaction pressure of 0.5 to 5.0 MPa for a reaction time of 1 to 50 h. More preferably, the reaction temperature is 0 to 50° C. the reaction pressure is 0.8 to 3.0 MPa, and the reaction time is 5 to 10 h.

In the dehydrohalogenation step of the present disclosure, under the action of the composite dehalogenation catalyst, the 3-chloro-1,1,1,2-tetrafluoropropane is subjected to the dehydrochlorination reaction when adsorbed to activated carbon and is subjected to the dehydrofluorination reaction when adsorbed to Al₂O₃ and/or AlF₃ and/or MgF₂ and/or Cr₂O₃, so as to obtain the 2,3,3,3-tetrafluoropropene and the 1-chloro-3,3,3-trifluoropropene at the same time.

The composite dehalogenation catalyst of the present disclosure may be prepared by a conventional method in a case that the composite dehalogenation catalyst of the present disclosure can be obtained. As a preference, the composite dehalogenation catalyst is prepared by a co-blending method. The co-blending method includes the following steps:
B1, mixing: blending Al₂O₃ and/or AlF₃ and/or MgF₂ and/or Cr₂O₃ with activated carbon powder at a mass ratio of (0.01-0.25):1 and performing thorough mixing by a mechanical stirring mode or a ball milling mode;
B2, sifting: sifting the mixed material to remove an unevenly mixed part;
B3, molding: transferring the sifted material to a tablet press for compression molding; and
B4, drying a molded catalyst to prepare the composite dehalogenation catalyst, such as Al₂O₃-AC, AlF₃-AC, MgF₂-AC, Cr₂O₃-AC and other catalysts.

In the molding step B3, the catalyst may be prepared in a columnar shape, a sheet shape and other shapes, and the specific shape is not limited.

In the step B4, the drying is usually carried out at 90 to 120° C. for 12 h or above.

As for the composite dehalogenation catalyst of the present disclosure, when the Al₂O₃ is co-blended with the activated carbon powder, the content of the Al₂O₃ is 1.0 to 20 wt % of the total amount of the catalyst; when the AlF₃ is co-blended with the activated carbon powder, the content of the AlF₃ is 1.0 to 20 wt % of the total amount of the catalyst; when the MgF₂ is co-blended with the activated carbon powder, the content of the MgF₂ is 1.0 to 20 wt % of the total amount of the catalyst; and when the Cr₂O₃ is co-blended with the activated carbon powder, the content of the Cr₂O₃ is 1.0 to 20 wt % of the total amount of the catalyst.

A gas-solid reaction is carried out in the dehydrohalogenation step of the present disclosure, the 3-chloro-1,1,1,2-tetrafluoropropane is vaporized and then loaded onto a catalyst bed layer by nitrogen to carry out a dehydrohalogenation reaction, the dehydrohalogenation reaction has a material volume space velocity of 50 to 300 h, and the volume ratio of N₂ to the 3-chloro-1,1,1,2-tetrafluoropropane is (0.5-3.0):1, preferably (1.5-2.0):1.

The dehydrohalogenation step of the present disclosure is carried out at a reaction temperature of 300 to 500° C., and preferably, the reaction temperature is 350-450° C.

Distribution of products obtained in the dehydrohalogenation step may be adjusted in a certain range by adjusting the preparation process of the composite dehalogenation catalyst, contents of active components in the catalyst and reaction conditions. Generally, 10 to 50% of the 2,3,3,3-tetrafluoropropene and 10-70% of the 1-chloro-3,3,3-trifluoropropene are obtained in the dehydrohalogenation step; and preferably, products obtained in the dehydrohalogenation step include 20-40% of the 2,3,3,3-tetrafluoropropene, 30-60% of the 1-chloro-3,3,3-trifluoropropene and remaining unknown by-products.

In order to further reduce post-treatment difficulty, the 3-chloro-1,1,1,2-tetrafluoropropane obtained in the telomerization step is used in the dehydrohalogenation step after rectification and separation.

Compared with the prior art, the present disclosure has the following beneficial effects.

According to the present disclosure, chlorofluoromethane and trifluoroethylene are used as raw materials and subjected to pressure telomerization under the action of a Lewis acid catalyst or a mixed catalyst of a Lewis acid catalyst and dichloromethane to obtain 3-chloro-1,1,1,2-tetrafluoropropane. The 3-chloro-1,1,1,2-tetrafluoropropane is prepared into 2,3,3,3-tetrafluoropropene under the catalytic action of activated carbon: or, the 3-chloro-1,1,1,2-tetrafluoropropane is subjected to a dehydrochlorination reaction and a dehydrogenation reaction simultaneously under the catalytic action of an activated carbon supported noble metal catalyst to co-produce 2,3,3,3-tetrafluoropropene and 1-chloro-2,3,3,3-tetrafluoropropene; or, the 3-chloro-1,1,1,2-tetrafluoropropane is subjected to a dehydrochlorination reaction and a dehydrofluorination reaction simultaneously under the catalytic action of a composite dehalogenation catalyst to co-produce 2,3,3,3-tetrafluoropropene and 1-chloro-3,3,3-trifluoropropene. The three methods for preparing 2,3,3,3-tetrafluoropropene provided by the present disclosure have the advantages of a simple process, mild reaction conditions, high selectivity of a telomerization product and a target product and the like, and are suitable for industrial amplification.

### DETAILED DESCRIPTION OF THE EMBODIMENTS

The present disclosure is further described below in combination with specific embodiments, but the present disclosure is not limited to these specific embodiments. For persons skilled in the art, it is to be understood that the present disclosure covers all possible alternative schemes, improved schemes and equivalent schemes included within the scope of the claims A first aspect of the embodiments of the present disclosure is to provide a two-step method for preparing 2,3,3,3-tetrafluoropropene.

### Example 1.1

The present example provides a two-step method for preparing 2,3,3,3-tetrafluoropropene. The method includes a telomerization step and a dehydrochlorination step and is specifically as follows.

### 1. Telomerization Step

A1. An autoclave made of an Inconel alloy with a volume of 250 mL was used as a reactor. 3.0 g of HfCl₄ and 20.0 g of dichloromethane were separately added into the reactor, then the reactor was sealed, and 1.0 MPa of nitrogen was repeatedly introduced to replace air in the reactor for three times.

A2. After the air in the reactor was completely replaced, 19.9 g (0.29 mol) of chlorofluoromethane and 24.6 g (0.30 mol) of trifluoroethylene were sequentially introduced.

A3. The reaction temperature was set at 10° C., the stirring rate was set at 300 rpm, and the initial reaction pressure was set at 0.9 MPa. With progressing of a reaction, the pressure was gradually decreased, and the reaction time was 10 h.

A4. After the reaction was completed, unreacted gas phase raw materials including the trifluoroethylene and/or the chlorofluoromethane and small amounts of a telomerization product and the dichloromethane were collected. The materials in the reactor were subjected to solid-liquid separation treatment such as filtration or distillation, wherein a Lewis acid catalyst (HfCl₄) was used as a solid part, and the dichloromethane and the telomerization product were used as liquid phase materials. Then, rectification and separation were carried out to obtain 3-chloro-1,1,1,2-tetrafluoropropane with a purity of 99.9% for use in a dehydrochlorination reaction.

The unreacted gas phase raw materials and the separated Lewis acid catalyst were transferred back to the telomerization step for reuse.

According to analysis of the gas phase and liquid phase materials by gas chromatography, the conversion rate of chlorofluoromethane was 76.5%, the selectivity of 3-chloro-1,1,1,2-tetrafluoropropane was 81.2%, 1-chloro-1,1,2,3-tetrafluoropropane was a main by-product with a selectivity of 15.3%, and few other by-products were produced.

### 2. Dehydrochlorination Step

B1. A reaction tube made of an Inconel alloy with an inner diameter of 19 mm and a length of 800 mm was used as a fixed bed reactor. Coconut shell type activated carbon with a volume of 20 mL and a particle size of 10-20 mesh was filled to the middle of the fixed bed reactor, a reaction pipeline was connected, and nitrogen was introduced for purging at a flow rate of 100 mL/min.

B2. The reaction temperature was set at 350° C. and the reactor was heated at a heating rate of 5° C./min.

B3. After a catalyst bed layer reached the reaction temperature, the nitrogen flow rate was adjusted to 20 mL/min. Meanwhile, the 3-chloro-1,1,1,2-tetrafluoropropane with a purity of 99.9% was continuously introduced into the fixed bed reactor at a rate of 5.0 g/h to carry out a reaction.

B4. According to analysis of a gas mixture flowing out of the reactor by on-line gas chromatography (GC) and gas chromatography-mass spectrometry (GC/MS), the conversion rate of 3-chloro-1,1,1,2-tetrafluoropropane was 99.6%, and the selectivity of a 2,3,3,3-tetrafluoropropene product was 99.3%.

### Example 1.2

The present example provides a method for preparing 2,3,3,3-tetrafluoropropene. The method has the same operations as that in Example 1.1, and only has the differences that in the telomerization step, 4.0 g of ZrCl₄ was used to replace the HfCl₄, the amount of chlorofluoromethane was increased to 39.7 g (0.58 mol) and the amount of trifluoroethylene was increased to 71.3 g (0.87 mol) while other conditions were remained unchanged.

According to analysis of gas phase and liquid phase materials in the telomerization step by gas chromatography, the conversion rate of chlorofluoromethane was 99.0%, the selectivity of 3-chloro-1,1,1,2-tetrafluoropropane was 89.9%, 1-chloro-1,1,2,3-tetrafluoropropane was a main by-product with a selectivity of 5.3%, and few other by-products were produced.

### Example 1.3

The present example provides a method for preparing 2,3,3,3-tetrafluoropropene. The method has the same operations as that in Example 1.2, and only has the differences that in the telomerization step, the dichloromethane was not used, the amount of trifluoroethylene was increased to 95.1 g (1.16 mol), meanwhile, the reaction temperature was increased to 30° C. and the initial reaction pressure was increased to 1.5 MPa while other conditions were remained unchanged.

According to analysis of gas phase and liquid phase materials in the telomerization step by gas chromatography, the conversion rate of chlorofluoromethane was 99.5%, the selectivity of 3-chloro-1,1,1,2-tetrafluoropropane was 88.1%, 1-chloro-1,1,2,3-tetrafluoropropane was a main by-product with a selectivity of 4.1%, and few other by-products were produced.

### Example 1.4

The present example provides a method for preparing 2,3,3,3-tetrafluoropropene. The method has the same operations as that in Example 1.2, and only has the differences that in the telomerization step, 4.0 g, the same use amount, of AlCl₃ was used to replace the ZrCl₄, the dichloromethane was not used and the amount of trifluoroethylene was decreased to 52.5 g (0.64 mol) while other conditions were remained unchanged.

According to analysis of gas phase and liquid phase materials in the telomerization step by gas chromatography, the conversion rate of chlorofluoromethane was 99.6%, the selectivity of 3-chloro-1,1,1,2-tetrafluoropropane was 75.5%, 1-chloro-1,1,2,3-tetrafluoropropane was a main by-product with a selectivity of 15.9%, and few other by-products were produced.

### Example 1.5

The present example provides a method for preparing 2,3,3,3-tetrafluoropropene. The method has the same operations as that in Example 1.1, and only has the differences that in the step A2 of the telomerization step, after the chlorofluoromethane and the trifluoroethylene were sequentially introduced into the autoclave, high-purity and high-pressure nitrogen was used to perform pressure treatment on the autoclave so as to increase the pressure in the autoclave from 0.9 MPa to 3.0 MPa while other conditions were remained unchanged.

According to analysis of gas phase and liquid phase materials in the telomerization step by gas chromatography, the conversion rate of chlorofluoromethane was 99.8%, the selectivity of 3-chloro-1,1,1,2-tetrafluoropropane was 88.6%, 1-chloro-1,1,2,3-tetrafluoropropane was a main by-product with a selectivity of 7.6%, and few other by-products were produced.

### Example 1.6

The present example provides a method for preparing 2,3,3,3-tetrafluoropropene. The method has the same operations as that in Example 1.1, and only has the difference that in the dehydrochlorination step, 10- to 20-mesh coal type activated carbon was used to replace the coconut shell type activated carbon.

According to analysis of a dehydrochlorination product by chromatography, the conversion rate of 3-chloro-1,1,1,2-tetrafluoropropane was 99.2%, and the selectivity of a 2,3,3,3-tetrafluoropropene product reached 95.1%.

### Example 1.7

The present example provides a method for preparing 2,3,3,3-tetrafluoropropene. The method has the same operations as that in Example 1.1, and only has the difference that in the dehydrochlorination step, the reaction temperature was lowered to 300° C.

According to analysis of a dehydrochlorination product by chromatography, the conversion rate of 3-chloro-1,1,1,2-tetrafluoropropane was 75.8%, and the selectivity of a 2,3,3,3-tetrafluoropropene product was 99.2%.

### Example 1.8

The present example provides a method for preparing 2,3,3,3-tetrafluoropropene. The method has the same operations as that in Example 1.1, and only has the difference that in the dehydrochlorination step, the reaction temperature was lowered to 320° C.

According to analysis of a dehydrochlorination product by chromatography, the conversion rate of 3-chloro-1,1,1,2-tetrafluoropropane was 86.9%, and the selectivity of a 2,3,3,3-tetrafluoropropene product was 99.1%.

### Comparative Example 1.1

The present comparative example provides a method for preparing 2,3,3,3-tetrafluoropropene. The method has the same operations as that in Example 1.1, and only has the difference that 20.0 g of trichloromethane was used to replace the dichloromethane while other conditions were remained unchanged.

According to analysis of materials obtained by a reaction in the telomerization step by chromatography, the conversion rate of chlorofluoromethane was 86.9%, the selectivity of 3-chloro-1,1,1,2-tetrafluoropropane was 46.2%, a large amount of dichloromethane, as a disproportionation product of the chlorofluoromethane, was produced with a selectivity of 40.3%, and few other telomerization by-products were produced.

### Comparative Example 1.2

The present comparative example provides a method for preparing 2,3,3,3-tetrafluoropropene. The method has the same operations as that in Example 1.1, and only has the difference that 3.0 g of ZnCl₂ was used to replace the HfCl₄ while other conditions were remained unchanged.

According to analysis of materials obtained by a reaction in the telomerization step by chromatography, the conversion rate of chlorofluoromethane was 20.8%, and a target product, 3-chloro-1,1,1,2-tetrafluoropropane, was not produced.

### Comparative Example 1.3

The present comparative example provides a method for preparing 2,3,3,3-tetrafluoropropene. The method has the same operations as that in Example 1.1, and only has the difference that the HfCl₄ and the dichloromethane were not added while other conditions were remained unchanged.

According to analysis of materials obtained by a reaction in the telomerization step by chromatography, the conversion rate of chlorofluoromethane was 7.7%, a target product, 3-chloro-1,1,1,2-tetrafluoropropane, was not produced, and only a small amount of dichloromethane, as a disproportionation product of the chlorofluoromethane, was produced.

A second aspect of the embodiments of the present disclosure is to provide a method for co-producing 2,3,3,3-tetrafluoropropene and 1-chloro-2,3,3,3-tetrafluoropropene.

### Preparative Example 2.1

6.0 mL of a chloropalladic acid solution (with a concentration of 0.033 g Pd/mL) was taken and added into 80.0 mL of distilled water for uniform dilution to obtain an impregnation solution. 20.0 g of activated carbon pretreated by drying at 120° C. for 12 h was taken and added into the impregnation solution for impregnation for 12 h or above, followed by drying at 120° C. for 12 h to obtain a 1 wt % Pd/AC catalyst, recorded as cat 2.1.

### Preparative Example 2.2

9.2 mL of a chloropalladic acid solution (with a concentration of 0.033 g Pd/mL) was taken and added into 80.0 mL of distilled water for uniform dilution to obtain an impregnation solution 20.0 g of activated carbon pretreated by drying at 120° C. for 12 h was taken and added into the impregnation solution for impregnation for 12 h or above, followed by drying at 120° C. for 12 h to obtain a 1.5 wt % Pd/AC catalyst, recorded as cat 2.2.

### Preparative Example 2.3

0.35 g of PtCl₄ was taken and dissolved in 80.0 mL of distilled water to obtain an impregnation solution. 20.0 g of activated carbon pretreated by drying at 120° C. for 12 h was taken and added into the impregnation solution for impregnation for 12 h or above, followed by drying at 120° C. for 12 h to obtain a 1 wt % Pt/AC catalyst, recorded as cat 2.3.

### Preparative Example 2.4

0.52 g of PtCl₄ was taken and dissolved in 80.0 mL of distilled water to obtain an impregnation solution. 20.0 g of activated carbon pretreated by drying at 120° C. for 12 h was taken and added into the impregnation solution for impregnation for 12 h or above, followed by drying at 120° C. for 12 h to obtain a 1.5 wt % Pt/AC catalyst, recorded as cat 2.4.

### Example 2.1

The present example provides a method for co-producing 2,3,3,3-tetrafluoropropene and 1-chloro-2,3,3,3-tetrafluoropropene. The method includes a telomerization step and a removal step and is specifically as follows.

### 1. Telomerization Step

A1. An autoclave made of an Inconel alloy with a volume of 250 mL was used as a reactor 3.0 g of HfCl₄ and 20.0 g of dichloromethane were separately added into the reactor, then the reactor was sealed, and 1.0 MPa of nitrogen was repeatedly introduced to replace air in the reactor for three times.

A2. After the air in the reactor was completely replaced, 19.9 g (0.29 mol) of chlorofluoromethane and 24.6 g (0.30 mol) of trifluoroethylene were sequentially introduced.

A3. The reaction temperature was set at 10° C., the stirring rate was set at 300 rpm, and the initial reaction pressure was set at 0.9 MPa. With progressing of a reaction, the pressure was gradually decreased, and the reaction time was 10 h.

A4. After the reaction was completed, unreacted gas phase raw materials including the trifluoroethylene and/or the chlorofluoromethane and small amounts of a telomerization product and the dichloromethane were collected. The materials in the reactor were subjected to solid-liquid separation treatment such as filtration or distillation, wherein a Lewis acid catalyst (HfCl₄) was used as a solid part, and the dichloromethane and the telomerization product were used as liquid phase materials. Then, rectification and separation were carried out to obtain 3-chloro-1,1,1,2-tetrafluoropropane with a purity of 99.9% for use in the removal step.

The unreacted gas phase raw materials and the separated Lewis acid catalyst were transferred back to the telomerization step for reuse.

According to analysis of the gas phase and liquid phase materials by gas chromatography, the conversion rate of chlorofluoromethane was 76.5%, the selectivity of 3-chloro-1,1,1,2-tetrafluoropropane was 81.2%, 1-chloro-1,1,2,3-tetrafluoropropane was a main by-product with a selectivity of 15.3%, and few other by-products were produced.

### 2. Removal Step

B1. A reaction tube made of an Inconel alloy with an inner diameter of 19 mm and a length of 800 mm was used as a fixed bed reactor. Cat 2.1 with a volume of 20 mL was filled to the middle of the fixed bed reactor, a reaction pipeline was connected, and nitrogen was introduced for purging at a flow rate of 100 mL/min.

B2 The reaction temperature was set at 450° C., and the reactor was heated at a heating rate of 5° C./min.

B3. After a catalyst bed layer reached the reaction temperature, the nitrogen flow rate was adjusted to 20 mL/min. Meanwhile, the 3-chloro-1,1,1,2-tetrafluoropropane with a purity of 99.9% was continuously introduced into the fixed bed reactor at a rate of 5.0 g/h by a peristaltic pump to carry out a reaction.

B4. A gas mixture flowing out of the reactor was subjected to heat preservation treatment, followed by analysis by on-line GC and GC/MS. The conversion rate of 3-chloro-1,1,1,2-tetrafluoropropane was 96.8%, the content of 2,3,3,3-tetrafluoropropene in the product was 56.3%, and the content of 1-chloro-2,3,3,3-tetrafluoropropene was 31.4%.

### Example 2.2

The present example provides a method for co-producing 2,3,3,3-tetrafluoropropene and 1-chloro-2,3,3,3-tetrafluoropropene. The method has the same operations as that in Example 2.1, and only has the differences that in the telomerization step, 4.0 g of ZrCl₄ was used to replace the HfCl₄, the amount of chlorofluoromethane was increased to 39.7 g (0.58 mol) and the amount of trifluoroethylene was increased to 71.3 g (0.87 mol) while other conditions were remained unchanged.

According to analysis of gas phase and liquid phase materials in the telomerization step by gas chromatography, the conversion rate of chlorofluoromethane was 99.0%, the selectivity of 3-chloro-1,1,1,2-tetrafluoropropane was 89.9%, 1-chloro-1,1,2,3-tetrafluoropropane was a main by-product with a selectivity of 5.3%, and few other by-products were produced.

### Example 2.3

The present example provides a method for co-producing 2,3,3,3-tetrafluoropropene and 1-chloro-2,3,3,3-tetrafluoropropene. The method has the same operations as that in Example 2.2, and only has the differences that in the telomerization step, the dichloromethane was not used, the amount of trifluoroethylene was increased to 95.1 g (1.16 mol), meanwhile, the reaction temperature was increased to 30° C. and the initial reaction pressure was increased to 1.5 MPa while other conditions were remained unchanged.

According to analysis of gas phase and liquid phase materials in the telomerization step by gas chromatography, the conversion rate of chlorofluoromethane was 99.5%, the selectivity of 3-chloro-1,1,1,2-tetrafluoropropane was 88.1%, 1-chloro-1,1,2,3-tetrafluoropropane was a main by-product with a selectivity of 4.1%, and few other by-products were produced.

### Example 2.4

The present example provides a method for co-producing 2,3,3,3-tetrafluoropropene and 1-chloro-2,3,3,3-tetrafluoropropene. The method has the same operations as that in Example 2.2, and only has the differences that in the telomerization step, 4 g, the same use amount, of AlCl₃ was used to replace the ZrCl₄, the dichloromethane was not used and the amount of trifluoroethylene was decreased to 52.5 g (0.64 mol) while other conditions were remained unchanged.

According to analysis of gas phase and liquid phase materials in the telomerization step by gas chromatography, the conversion rate of chlorofluoromethane was 99.6%, the selectivity of 3-chloro-1,1,1,2-tetrafluoropropane was 75.5%, 1-chloro-1,1,2,3-tetrafluoropropane was a main by-product with a selectivity of 15.9%, and few other by-products were produced.

### Example 2.5

The present example provides a method for co-producing 2,3,3,3-tetrafluoropropene and 1-chloro-2,3,3,3-tetrafluoropropene. The method has the same operations as that in Example 2.1, and only has the differences that in the step A2 of the telomerization step, after the chlorofluoromethane and the trifluoroethylene were introduced into the autoclave in advance, high-purity and high-pressure nitrogen was used to perform pressure treatment on the autoclave so as to increase the pressure in the autoclave from 0.9 MPa to 3.0 MPa while other conditions were remained unchanged.

According to analysis of gas phase and liquid phase materials in the telomerization step by gas chromatography, the conversion rate of chlorofluoromethane was 99.8%, the selectivity of 3-chloro-1,1,1,2-tetrafluoropropane was 88.6%, 1-chloro-1,1,2,3-tetrafluoropropane was a main by-product with a selectivity of 7.6%, and few other by-products were produced.

### Example 2.6

The present example provides a method for co-producing 2,3,3,3-tetrafluoropropene and 1-chloro-2,3,3,3-tetrafluoropropene. The method has the same operations as that in Example 2.1, and only has the difference that in the removal step, cat 2.3 was used to replace the cat 2.1.

According to analysis of a removal reaction product by chromatography, the conversion rate of 3-chloro-1,1,1,2-tetrafluoropropane was 89.3%, the content of 2,3,3,3-tetrafluoropropene in the product was 90.3%, and the content of 1-chloro-2,3,3,3-tetrafluoropropene was 7.7%.

### Example 2.7

The present example provides a method for co-producing 2,3,3,3-tetrafluoropropene and 1-chloro-2,3,3,3-tetrafluoropropene. The method has the same operations as that in Example 2.1, and only has the difference that in the removal step, the amount of the cat 2.1 was increased to 40 mL.

According to analysis of a removal reaction product by chromatography, the conversion rate of 3-chloro-1,1,1,2-tetrafluoropropane was 95.8%, the content of 2,3,3,3-tetrafluoropropene in the product was 65.7%, and the content of 1-chloro-2,3,3,3-tetrafluoropropene was 26.8%.

### Example 2.8

The present example provides a method for co-producing 2,3,3,3-tetrafluoropropene and 1-chloro-2,3,3,3-tetrafluoropropene. The method has the same operations as that in Example 2.1, and only has the difference that in the removal step, cat 2.2 was used to replace the cat 2.1.

According to analysis of a removal reaction product by chromatography, the conversion rate of 3-chloro-1,1,1,2-tetrafluoropropane was 70.1%, the content of 2,3,3,3-tetrafluoropropene in the product was 39.2%, and the content of 1-chloro-2,3,3,3-tetrafluoropropene was 28.5%.

### Example 2.9

The present example provides a method for co-producing 2,3,3,3-tetrafluoropropene and 1-chloro-2,3,3,3-tetrafluoropropene. The method has the same operations as that in Example 2.1, and only has the difference that in the removal step, the reaction temperature was 400° C.

According to analysis of a removal reaction product by chromatography, the conversion rate of 3-chloro-1,1,1,2-tetrafluoropropane was 96.7%, the content of 2,3,3,3-tetrafluoropropene in the product was 85.2%, and the content of 1-chloro-2,3,3,3-tetrafluoropropene was 10.3%.

### Comparative Example 2.1

The present comparative example provides a method for co-producing 2,3,3,3-tetrafluoropropene and 1-chloro-2,3,3,3-tetrafluoropropene. The method has the same operations as that in Example 2.1, and only has the difference that 20.0 g of trichloromethane was used to replace the dichloromethane while other conditions were remained unchanged.

According to analysis of materials obtained by a reaction in the telomerization step by chromatography, the conversion rate of chlorofluoromethane was 86.9%, the selectivity of 3-chloro-1,1,1,2-tetrafluoropropane was 46.2%, a large amount of dichloromethane, as a disproportionation product of the chlorofluoromethane, was produced with a selectivity of 40.3%, and few other telomerization by-products were produced.

### Comparative Example 2.2

The present comparative example provides a method for co-producing 2,3,3,3-tetrafluoropropene and 1-chloro-2,3,3,3-tetrafluoropropene. The method has the same operations as that in Example 2.1, and only has the difference that 3.0 g of ZnCl₂ was used to replace the HfCl₄ while other conditions were remained unchanged.

According to analysis of materials obtained by a reaction in the telomerization step by chromatography, the conversion rate of chlorofluoromethane was 20.8%, and a target product, 3-chloro-1,1,1,2-tetrafluoropropane, was not produced.

### Comparative Example 2.3

The present comparative example provides a method for co-producing 2,3,3,3-tetrafluoropropene and 1-chloro-2,3,3,3-tetrafluoropropene. The method has the same operations as that in Example 2.1, and only has the difference that the HfCl₄ and the dichloromethane were not added while other conditions were remained unchanged.

According to analysis of materials obtained by a reaction in the telomerization step by chromatography, the conversion rate of chlorofluoromethane was 7.7%, a target product, 3-chloro-1,1,1,2-tetrafluoropropane, was not produced, and only a small amount of dichloromethane, as a disproportionation product of the chlorofluoromethane, was produced.

### Comparative Example 2.4

The present comparative example provides a method for co-producing 2,3,3,3-tetrafluoropropene and 1-chloro-2,3,3,3-tetrafluoropropene. The method has the same operations as that in Example 2.1, and only has the difference that in the removal step, activated carbon pretreated by drying at 120° C. for 12 h was used to replace the cat 2.1 while other conditions were remained unchanged.

According to analysis of a removal reaction product by chromatography, the conversion rate of 3-chloro-1,1,1,2-tetrafluoropropane was 99.7%, the content of 2,3,3,3-tetrafluoropropene in the product was 99.0%, and 1-chloro-2,3,3,3-tetrafluoropropene was not produced.

### Comparative Example 2.5

The present comparative example provides a method for co-producing 2,3,3,3-tetrafluoropropene and 1-chloro-2,3,3,3-tetrafluoropropene. The method has the same operations as that in Example 2.1, and only has the difference that Al₂O₃ was used to replace the cat 2.1 while other conditions were remained unchanged.

According to analysis of a removal reaction product by chromatography, the conversion rate of 3-chloro-1,1,1,2-tetrafluoropropane was 50.1%, the content of 2,3,3,3-tetrafluoropropene in the product was 3.1%, the content of 1-chloro-3,3,3-trifluoropropene was 62.2%, and 1-chloro-2,3,3,3-tetrafluoropropene was not produced.

A third aspect of the embodiments of the present disclosure is to provide a method for co-producing 2,3,3,3-tetrafluoropropene and 1-chloro-3,3,3-trifluoropropene.

### Preparative Example 3.1

The present preparative example provides preparation of a Cr₂O₃-AC catalyst by co-blending Cr₂O₃ with activated carbon powder. The preparation includes the following steps:
S1, blending Cr₂O and coconut shell type activated carbon powder at a mass ratio of 1/9, and placing the blended material into a ball mill for ball milling and mixing so as to achieve even dispersion of various components;
S2, sifting the mixed material to remove an unevenly mixed part;
S3, transferring the sifted material to a tablet press for compression molding to obtain a columnar catalyst; and
S4, drying the molded catalyst at 120° C. for 12 h to prepare a Cr₂O₃-AC catalyst, recorded as cat 3.1.

### Preparative Example 3.2

The present preparative example has the same operations as that in Preparative Example 3.1, and only has the differences that AlF₃ was used to replace the Cr₂O₃, and an AlF₃-AC catalyst was prepared, which was recorded as cat 3.2.

### Preparative Example 3.3

The present preparative example has the same operations as that in Preparative Example 3.1, and only has the differences that the mass ratio of the Cr₂O₃ to the activated carbon was changed into 1/4, and a Cr₂O₃-AC catalyst was prepared, which was recorded as cat 3.3.

### Example 3.1

The present example provides a method for co-producing 2,3,3,3-tetrafluoropropene and 1-chloro-3,3,3-trifluoropropene. The method includes a telomerization step and a dehydrohalogenation step and is specifically as follows.

### 1. Telomerization Step

A1. An autoclave made of an Inconel alloy with a volume of 250 mL was used as a reactor, 3.0 g of HfCl₄ and 20.0 g of dichloromethane were separately added into the reactor, then the reactor was sealed, and 1.0 MPa of nitrogen was repeatedly introduced to replace air in the reactor for three times.

A2. After the air in the reactor was completely replaced, 19.9 g (0.29 mol) of chlorofluoromethane and 24.6 g (0.30 mol) of trifluoroethylene were sequentially introduced.

A3. The reaction temperature was set at 10° C., the stirring rate was set at 300 rpm, and the initial reaction pressure was set at 0.9 MPa. With progressing of a reaction, the pressure was gradually decreased, and the reaction time was 10 h.

A4. After the reaction was completed, unreacted gas phase raw materials including the trifluoroethylene and/or the chlorofluoromethane and small amounts of a telomerization product and the dichloromethane were collected. The materials in the reactor were subjected to solid-liquid separation treatment such as filtration or distillation, wherein a Lewis acid catalyst (HfCl₄) was used as a solid part, and the dichloromethane and the telomerization product were used as liquid phase materials. Then, rectification and separation were carried out to obtain 3-chloro-1,1,1,2-tetrafluoropropane with a purity of 99.9% for use in the dehydrohalogenation step.

The unreacted gas phase raw materials and the separated Lewis acid catalyst were transferred back to the telomerization step for reuse.

According to analysis of the gas phase and liquid phase materials by gas chromatography, the conversion rate of chlorofluoromethane was 76.5%, the selectivity of 3-chloro-1,1,1,2-tetrafluoropropane was 81.2%, 1-chloro-1,1,2,3-tetrafluoropropane was a main by-product with a selectivity of 15.3%, and few other by-products were produced.

### 2. Dehydrohalogenation Step

B1. A reaction tube made of an Inconel alloy with an inner diameter of 19 mm and a length of 800 mm was used as a fixed bed reactor. Cat 3.1 with a volume of 20 mL was filled to the middle of the fixed bed reactor, a reaction pipeline was connected, and nitrogen was introduced for purging at a flow rate of 100 mL/min.

B2. The reaction temperature was set at 350° C., and the reactor was heated at a heating rate of 5° C./min.

B3. After a catalyst bed layer reached the reaction temperature, the nitrogen flow rate was adjusted to 20 mL/min. Meanwhile, the 3-chloro-1,1,1,2-tetrafluoropropane with a purity of 99.9% was continuously introduced into the fixed bed reactor at a rate of 5.0 g/h to carry out a reaction.

B4. A gas mixture flowing out of the reactor was subjected to heat preservation treatment, followed by analysis by on-line GC and GC/MS. The conversion rate of 3-chloro-1,1,1,2-tetrafluoropropane was 88.7%, the content of 2,3,3,3-tetrafluoropropene in the product was 24.1%, and the content of 1-chloro-3,3,3-trifluoropropene was 58.7%.

### Example 3.2

The present example provides a method for co-producing 2,3,3,3-tetrafluoropropene and 1-chloro-3,3,3-trifluoropropene. The method has the same operations as that in Example 3.1, and only has the differences that in the telomerization step, 4.0 g of ZrCl₄ was used to replace the HfCl₄, the amount of chlorofluoromethane was increased to 39.7 g (0.58 mol) and the amount of trifluoroethylene was increased to 71.3 g (0.87 mol) while other conditions were remained unchanged.

According to analysis of gas phase and liquid phase materials in the telomerization step by gas chromatography, the conversion rate of chlorofluoromethane was 99.0%, the selectivity of 3-chloro-1,1,1,2-tetrafluoropropane was 89.9%, 1-chloro-1,1,2,3-tetrafluoropropane was a main by-product with a selectivity of 5.3%, and few other by-products were produced.

### Example 3.3

The present example provides a method for co-producing 2,3,3,3-tetrafluoropropene and 1-chloro-3,3,3-trifluoropropene. The method has the same operations as that in Example 3.2, and only has the differences that in the telomerization step, the dichloromethane was not used, the amount of trifluoroethylene was increased to 95.1 g (1.16 mol), meanwhile, the reaction temperature was increased to 30° C. and the initial reaction pressure was increased to 1.5 MPa while other conditions were remained unchanged.

According to analysis of gas phase and liquid phase materials in the telomerization step by gas chromatography, the conversion rate of chlorofluoromethane was 99.5%, the selectivity of 3-chloro-1,1,1,2-tetrafluoropropane was 88.1%, 1-chloro-1,1,2,3-tetrafluoropropane was a main by-product with a selectivity of 4.1%, and few other by-products were produced.

### Example 3.4

The present example provides a method for co-producing 2,3,3,3-tetrafluoropropene and 1-chloro-3,3,3-trifluoropropene. The method has the same operations as that in Example 3.2, and only has the differences that in the telomerization step, 4.0 g. the same use amount, of AlCl₃ was used to replace the ZrCl₄, the dichloromethane was not used and the amount of trifluoroethylene was decreased to 52.5 g (0.64 mol) while other conditions were remained unchanged.

According to analysis of gas phase and liquid phase materials in the telomerization step by gas chromatography, the conversion rate of chlorofluoromethane was 99.6%, the selectivity of 3-chloro-1,1,1,2-tetrafluoropropane was 75.5%. 1-chloro-1,1,2,3-tetrafluoropropane was a main by-product with a selectivity of 15.9%, and few other by-products were produced.

### Example 3.5

The present example provides a method for co-producing 2,3,3,3-tetrafluoropropene and 1-chloro-3,3,3-trifluoropropene. The method has the same operations as that in Example 3.1, and only has the differences that in the step A2 of the telomerization step, after the chlorofluoromethane and the trifluoroethylene were sequentially introduced into the autoclave, high-purity and high-pressure nitrogen was used to perform pressure treatment on the autoclave so as to increase the pressure in the autoclave from 0.9 MPa to 3.0 MPa while other conditions were remained unchanged.

According to analysis of gas phase and liquid phase materials in the telomerization step by gas chromatography, the conversion rate of chlorofluoromethane was 99.8%, the selectivity of 3-chloro-1,1,1,2-tetrafluoropropane was 88.6%, 1-chloro-1,1,2,3-tetrafluoropropane was a main by-product with a selectivity of 7.6%, and few other by-products were produced.

### Example 3.6

The present example provides a method for co-producing 2,3,3,3-tetrafluoropropene and 1-chloro-3,3,3-trifluoropropene. The method has the same operations as that in Example 3.1, and only has the difference that in the dehydrohalogenation step, cat 3.2 was used to replace the cat 3.1.

According to analysis of a dehydrohalogenation reaction product by chromatography, the conversion rate of 3-chloro-1,1,1,2-tetrafluoropropane was 92.9%, the content of 2,3,3,3-tetrafluoropropene in the product was 20.3%, and the content of 1-chloro-3,3,3-trifluoropropene was 58.7%.

### Example 3.7

The present example provides a method for co-producing 2,3,3,3-tetrafluoropropene and 1-chloro-3,3,3-trifluoropropene. The method has the same operations as that in Example 3.1, and only has the difference that in the dehydrohalogenation step, the amount of the cat 3.1 was increased to 40 mL.

According to analysis of a dehydrohalogenation reaction product by chromatography, the conversion rate of 3-chloro-1,1,1,2-tetrafluoropropane was greater than 95.9%, the content of 2,3,3,3-tetrafluoropropene in the product was 16.1%, and the content of 1-chloro-3,3,3-trifluoropropene was 44.6%.

### Example 3.8

The present example provides a method for co-producing 2,3,3,3-tetrafluoropropene and 1-chloro-3,3,3-trifluoropropene. The method has the same operations as that in Example 3.1, and only has the difference that in the dehydrohalogenation step, the reaction temperature was 450° C.

According to analysis of a dehydrohalogenation reaction product by chromatography, the conversion rate of 3-chloro-1,1,1,2-tetrafluoropropane was 98.3%, the content of 2,3,3,3-tetrafluoropropene in the product was 15.9%, and the content of 1-chloro-3,3,3-trifluoropropene was 60.0%.

### Comparative Example 3.1

The present comparative example provides a method for co-producing 2,3,3,3-tetrafluoropropene and 1-chloro-3,3,3-trifluoropropene. The method has the same operations as that in Example 3.1, and only has the difference that 20 g of trichloromethane was used to replace the dichloromethane while other conditions were remained unchanged.

According to analysis of materials obtained by a reaction in the telomerization step by chromatography, the conversion rate of chlorofluoromethane was 86.9%, the selectivity of 3-chloro-1,1,1,2-tetrafluoropropane was 46.1%, a large amount of dichloromethane, as a disproportionation product of the chlorofluoromethane, was produced with a selectivity of 40.3%, and few other telomerization by-products were produced.

### Comparative Example 3.2

The present comparative example provides a method for co-producing 2,3,3,3-tetrafluoropropene and 1-chloro-3,3,3-trifluoropropene. The method has the same operations as that in Example 3.1, and only has the difference that 3.0 g of ZnCl₂ was used to replace the HfCl₄ while other conditions were remained unchanged.

According to analysis of materials obtained by a reaction in the telomerization step by chromatography, the conversion rate of chlorofluoromethane was 20.8%, and a target product, 3-chloro-1,1,1,2-tetrafluoropropane, was not produced.

### Comparative Example 3.3

The present comparative example provides a method for co-producing 2,3,3,3-tetrafluoropropene and 1-chloro-3,3,3-trifluoropropene. The method has the same operations as that in Example 3.1, and only has the difference that the HfCl₄ and the dichloromethane were not added while other conditions were remained unchanged.

According to analysis of materials obtained by a reaction in the telomerization step by chromatography, the conversion rate of chlorofluoromethane was 7.6%, a target product, 3-chloro-1,1,1,2-tetrafluoropropane, was not produced, and only a small amount of dichloromethane, as a disproportionation product of the chlorofluoromethane, was produced.

### Comparative Example 3.4

The present comparative example provides a method for co-producing 2,3,3,3-tetrafluoropropene and 1-chloro-3,3,3-trifluoropropene. The method has the same operations as that in Example 3.1, and only has the difference that in the dehydrohalogenation step, coconut shell type activated carbon pretreated by drying at 120° C. for 12 h was used to replace the cat 3.1 while other conditions were remained unchanged.

According to analysis of a dehydrohalogenation reaction product by chromatography, the conversion rate of 3-chloro-1,1,1,2-tetrafluoropropane was greater than 99.7%, the content of 2,3,3,3-tetrafluoropropene in the product was 99.0%, and 1-chloro-3,3,3-trifluoropropene was not produced.

### Comparative Example 3.5

The present comparative example provides a method for co-producing 2,3,3,3-tetrafluoropropene and 1-chloro-3,3,3-trifluoropropene. The method has the same operations as that in Example 3.1, and only has the difference that a Pd/AC catalyst with a Pd supporting capacity of 1 wt % was used to replace the cat 3.1 while other conditions were remained unchanged.

According to analysis of a dehydrohalogenation reaction product by chromatography, the conversion rate of 3-chloro-1,1,1,2-tetrafluoropropane was 83.5%, the content of 2,3,3,3-tetrafluoropropene in the product was 96.4%, the content of 1-chloro-2,3,3,3-tetrafluoropropene was 1.3%, and 1-chloro-3,3,3-trifluoropropene was not produced.

What is claimed is:

1. A two-step method for preparing 2,3,3,3-tetrafluoropropene, comprising:
A1, a telomerization step: subjecting chlorofluoromethane and trifluoroethylene to a pressure telomerization reaction under the action of a telomerization catalyst to prepare 3-chloro-1,1,1,2-tetrafluoropropane, wherein the telomerization catalyst is a Lewis acid catalyst or a mixed catalyst of a Lewis acid catalyst and dichloromethane; and
A2, a dehydrochlorination step: subjecting the 3-chloro-1,1,1,2-tetrafluoropropane to dehydrochlorination under the catalytic action of activated carbon to obtain 2,3,3,3-tetrafluoropropene; the activated carbon is selected from fruit shell type activated carbon, coal type activated carbon or wood type activated carbon.

2. The two-step method for preparing 2,3,3,3-tetrafluoropropene according to claim 1, wherein the Lewis acid catalyst is selected from at least one halide of Al, Sb, Ti, Zr and Hf.

3. The two-step method for preparing 2,3,3,3-tetrafluoropropene according to claim 2, wherein the Lewis acid catalyst is selected from at least one of ZrCl₄, HfCl₄, TiCl₄, AlCl₃, AlF₃ and SbF₅.

4. The two-step method for preparing 2,3,3,3-tetrafluoropropene according to claim 1, wherein the molar ratio of the chlorofluoromethane to the trifluoroethylene is 1:0.1 to 1:10;
the Lewis acid catalyst is 0.01 to 50 wt % of the mass of the chlorofluoromethane;
the molar ratio of the dichloromethane to the chlorofluoromethane is 1:0.01 to 1:10;
the pressure telomerization reaction is carried out at a temperature of −30° C. to 100° C. and a pressure of 0.5 to 5.0 MPa for 1 to 50 h.

5. (canceled)

6. (canceled)

7. (canceled)

8. (canceled)

9. The two-step method for preparing 2,3,3,3-tetrafluoropropene according to claim 1, wherein the dehydrochlorination step is carried out at a reaction temperature of 200 to 500° C.; the 3-chloro-1,1,1,2-tetrafluoropropane obtained in the telomerization step is used in the dehydrochlorination step after rectification and separation.

10. (canceled)

11. A method for co-producing 2,3,3,3-tetrafluoropropene and 1-chloro-2,3,3,3-tetrafluoropropene, comprising:
A1, a telomerization step: subjecting chlorofluoromethane and trifluoroethylene to a pressure telomerization reaction under the action of a telomerization catalyst to prepare 3-chloro-1,1,1,2-tetrafluoropropane, wherein the telomerization catalyst is a Lewis acid catalyst or a mixed catalyst of a Lewis acid catalyst and dichloromethane; and
A2, a removal step: subjecting the 3-chloro-1,1,1,2-tetrafluoropropane to a dehydrochlorination reaction and a dehydrogenation reaction simultaneously under the action of an activated carbon supported noble metal catalyst to obtain 2,3,3,3-tetrafluoropropene and 1-chloro-2,3,3,3-tetrafluoropropene, wherein the activated carbon supported noble metal catalyst is at least one of Pd/activated carbon and Pt/activated carbon; in the activated carbon supported noble metal catalyst, the Pd or the Pt has a supporting capacity of 0.1 to 5.0 wt %, and 30-90% of the 2,3,3,3-tetrafluoropropene and 10 to 50% of the 1-chloro-2,3,3,3-tetrafluoropropene are obtained in the removal step A2.

12. The method for co-producing 2,3,3,3-tetrafluoropropene and 1-chloro-2,3,3,3-tetrafluoropropene according to claim 11, wherein the Lewis acid catalyst is selected from at least one halide of Al, Sb, Ti, Zr and Hf.

13. The method for co-producing 2,3,3,3-tetrafluoropropene and 1-chloro-2,3,3,3-tetrafluoropropene according to claim 12, wherein the Lewis acid catalyst is selected from at least one of ZrCl₄, HfCl₄, TiCl₄, AlF₃, AlCl₃ and SbF₅.

14. The method for co-producing 2,3,3,3-tetrafluoropropene and 1-chloro-2,3,3,3-tetrafluoropropene according to claim 11, wherein the molar ratio of the chlorofluoromethane to the trifluoroethylene is 1:0.1 to 1:10; the Lewis acid catalyst is 0.01 to 50 wt % of the mass of the chlorofluoromethane; the molar ratio of the dichloromethane to the chlorofluoromethane is 1:0.01 to 1:10; the pressure telomerization reaction is carried out at a temperature of −30° C. to 100° C. and a pressure of 0.5 to 5.0 MPa for 1 to 50 h.

15. (canceled)

16. (canceled)

17. (canceled)

18. The method for co-producing 2,3,3,3-tetrafluoropropene and 1-chloro-2,3,3,3-tetrafluoropropene according to claim 11, wherein the activated carbon supported noble metal catalyst is prepared by an impregnation method, and the impregnation method comprises the following steps:
B1, pretreatment of a carrier: drying activated carbon at 90 to 120° C. for 12 h or above;
B2, impregnation in a metal salt: impregnating the pretreated activated carbon in a soluble salt solution of Pd or Pt under vacuum or atmospheric pressure conditions;
B3, drying the impregnated activated carbon at a temperature of 90 to 120° C. for 12 h or above; and
B4, reducing the dried activated carbon by a mixed gas of hydrogen and nitrogen to obtain the activated carbon supported noble metal catalyst, wherein the hydrogen has a volume ratio of 5 to 50% in the mixed gas of hydrogen and nitrogen, and the reducing is carried out at a temperature of 150 to 300° C.

19. (canceled)

20. The method for co-producing 2,3,3,3-tetrafluoropropene and 1-chloro-2,3,3,3-tetrafluoropropene according to claim 11, wherein the 3-chloro-1,1,1,2-tetrafluoropropane is vaporized and then loaded onto a catalyst bed layer by nitrogen to carry out a removal reaction, the removal reaction has a material volume space velocity of 50 to 300 h⁻¹, and the volume ratio of N₂ to the 3-chloro-1,1,1,2-tetrafluoropropane is (0.5-3.0):1; the removal step is carried out at a reaction temperature of 300 to 600° C.; the 3-chloro-1,1,1,2-tetrafluoropropane obtained in the telomerization step is used in the removal step after rectification and separation.

21. (canceled)

22. (canceled)

23. A method for co-producing 2,3,3,3-tetrafluoropropene and 1-chloro-3,3,3-trifluoropropene, comprising:
A1, a telomerization step: subjecting chlorofluoromethane and trifluoroethylene to a pressure telomerization reaction under the action of a telomerization catalyst to prepare 3-chloro-1,1,1,2-tetrafluoropropane, wherein the telomerization catalyst is a Lewis acid catalyst or a mixed catalyst of a Lewis acid catalyst and dichloromethane; and
A2, a dehydrohalogenation step: subjecting the 3-chloro-1,1,1,2-tetrafluoropropane to a dehydrochlorination reaction and a dehydrofluorination reaction simultaneously under the action of a composite dehalogenation catalyst to obtain 2,3,3,3-tetrafluoropropene and 1-chloro-3,3,3-trifluoropropene; the composite dehalogenation catalyst is prepared from at least one oxide or fluoride of Al, Mo or Cr and activated carbon powder; the activated carbon powder is selected from fruit shell type activated carbon, coal type activated carbon or wood type activated carbon.

24. The method for co-producing 2,3,3,3-tetrafluoropropene and 1-chloro-3,3,3-trifluoropropene according to claim 23, wherein the Lewis acid catalyst is selected from at least one halide of Al, Sb, Ti, Zr and Hf.

25. The method for co-producing 2,3,3,3-tetrafluoropropene and 1-chloro-3,3,3-trifluoropropene according to claim 24, wherein the Lewis acid catalyst is selected from at least one of ZrCl₄, HfCl₄, TiCl₄, AlF₃, AlCl₃ and SbF₅.

26. The method for co-producing 2,3,3,3-tetrafluoropropene and 1-chloro-3,3,3-trifluoropropene according to claim 23, wherein the molar ratio of the chlorofluoromethane to the trifluoroethylene is 1.0.1 to 1:10; the Lewis acid catalyst is 0.01 to 50 wt % of the mass of the chlorofluoromethane; the molar ratio of the dichloromethane to the chlorofluoromethane is 1:0.01 to 1:10; the pressure telomerization reaction is carried out at a temperature of −30° C. to 100° C. and a pressure of 0.5 to 5.0 MPa for 1 to 50 h.

27. (canceled)

28. (canceled)

29. (canceled)

30. (canceled)

31. The method for co-producing 2,3,3,3-tetrafluoropropene and 1-chloro-3,3,3-trifluoropropene according to claim 23, wherein the at least one oxide or fluoride of Al, Mg or Cr is selected from at least one of Al₂O₃, AlF₃, MgF₂ and Cr₂O₃.

32. (canceled)

33. The method for co-producing 2,3,3,3-tetrafluoropropene and 1-chloro-3,3,3-trifluoropropene according to claim 31, wherein the content of the Al₂O is 1.0 to 20 wt % of the total amount of the catalyst, the content of the AlF₃ is 1.0 to 20 wt % of the total amount of the catalyst, the content of the MgF₂ is 1.0 to 20 wt % of the total amount of the catalyst, and the content of the Cr₂O₃ is 1.0 to 20 wt % of the total amount of the catalyst.

34. The method for co-producing 2,3,3,3-tetrafluoropropene and 1-chloro-3,3,3-trifluoropropene according to claim 23, wherein the 3-chloro-1,1,1,2-tetrafluoropropane is vaporized and then loaded onto a catalyst bed layer by nitrogen to carry out a dehydrohalogenation reaction, the dehydrohalogenation reaction has a material volume space velocity of 50 to 300 h⁻¹, and the volume ratio of N₂ to the 3-chloro-1,1,1,2-tetrafluoropropane is (0.5-3.0):1; the dehydrohalogenation step is carried out at a reaction temperature of 300 to 500° C.; the 3-chloro-1,1,1,2-tetrafluoropropane obtained in the telomerization step is used in the dehydrohalogenation step after rectification and separation.

35. The method for co-producing 2,3,3,3-tetrafluoropropene and 1-chloro-3,3,3-trifluoropropene according to claim 23, wherein 10 to 50% of the 2,3,3,3-tetrafluoropropene and 10-70% of the 1-chloro-3,3,3-trifluoropropene are obtained in the dehydrohalogenation step.

36. The method for co-producing 2,3,3,3-tetrafluoropropene and 1-chloro-3,3,3-trifluoropropene according to claim 23, wherein the composite dehalogenation catalyst is prepared by a co-blending method, and the co-blending method comprises the following steps:
B1, mixing: blending Al₂O₃ and/or AlF₃ and/or MgF₂ and/or Cr₂O with activated carbon powder at a mass ratio of (0.01-0.25):1 and performing thorough mixing by a mechanical stirring mode or a ball milling mode;
B2, sifting: sifting the mixed material to remove an unevenly mixed part,
B3, molding: transferring the sifted material to a tablet press for compression molding; and
B4, drying a molded catalyst to prepare the composite dehalogenation catalyst.

37. (canceled)

38. (canceled)
